(19) United States
(12) Patent Application Publication
(10) Pub. No.: US 2011/0288014 A1
(43) Pub. Date: Nov. 24, 2011

(54) METHODS AND COMPOSITIONS FOR DIABETES TREATMENT AND PREVENTION

(75) Inventors: Thomas Hofmann, Neufahrn (DE); Sandra Held, Munich (DE); Roman Lang, Freising (DE); Veronika Somoza, Weidling (AU)

(73) Assignee: TUM Technische Universitat Munchen, Munich (DE)

(21) Appl. No.: 13/129,379

(22) PCT Filed: Nov. 17, 2009
(86) PCT No.: PCT/EP2009/065302

§ 371 Date: Aug. 4, 2011

Related U.S. Application Data
(60) Provisional application No. 61/115,116, filed on Nov. 17, 2008.

(51) Int. Cl. A61K 31/4425; A61P 3/10; A61K 38/28

(52) U.S. Cl. 514/6.5; 514/358; 514/354

(57) ABSTRACT

The present invention relates to methods and compositions for preventing or treating diabetes. The invention in particular discloses compounds according to formula (I) for use in the prevention and treatment of type II or I diabetes.

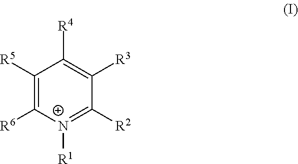

### FIELD OF THE INVENTION

The present invention relates to methods and compositions for preventing or treating diabetes.

### BACKGROUND OF THE INVENTION

Diabetes mellitus is a world-wide health problem and its incidence is increasing rapidly. In 2000, according to the World Health Organization, at least 171 million people worldwide suffer from diabetes, or 2.8% of the population, and it is estimated that by the year 2030, this number will almost double. For at least 20 years, diabetes rates in North America have been increasing substantially. In 2005 there were about 20.8 million people with diabetes in the United States alone. Diabetes mellitus prevalence increases with age, and the numbers of older persons with diabetes are expected to grow as the elderly population increases in number. Although various treatments are available, diabetes mellitus currently remains a chronic disease, without a cure, and thus there is a need for additional methods for treating and/or preventing this disease.

Recent epidemiological evidences show that moderate coffee consumption is associated with reduced risk of type II diabetes in humans and such risk reduction, up to 50%, is not related to caffeine consumption [Bidel et al. 2006, Diabetologia. 49: 2618-26; Greenberg et al. 2006, Am J Clin Nutr 84:682-93; Hiltunen 2006, Eur J Clin Nutr1; Paynter et al. 2006, Am J Epidemiol.; Pereira et al. 2006, Arch Intern Med. 166:1311-6; van Dam & Freskens 2002, Lancet 360, 1477-8; van Dam & Hu 2005, JAMA 294: 97-104]. However, the mechanism by which coffee beverages or their component(s) decrease blood glucose levels has not been identified yet. Elucidation of this mechanism or the identification of specific component(s) in coffee beverages responsible for this beneficial effect will no doubt lead to novel compositions or methods of diabetes prevention or treatment.

### DESCRIPTION OF THE INVENTION

The present invention discloses the identity of an active chemical component, N-methylpyridinium (N-MP) and derivatives thereof which have now been surprisingly discovered to have an insulin-like, or even insulin-enhancing, effect that affects the glucose uptake by adipocytes, one of the major mechanisms for lowering the blood glucose level.

One of the hallmarks of type I and type II diabetes is lack of sufficient insulin secretion from the pancreas, resulting in increased blood glucose levels. Upon insulin treatment, insulin receptors are activated which activates a signaling pathway leading to increased glucose uptake in adipocytes or muscle cells. Glucose uptake is, therefore, a very relevant end point assay in determining insulin sensitivity.

Specifically, the present inventors found that 2-deoxyglucose uptake in adipocytes in culture is increased if the cells are treated with either dark roast coffee (naturally high in N-MP), coffee spiked with N-MP, or N-MP as purified compound compared to control cells. Accordingly, in one embodiment, the present invention provides a method for preventing or treating diabetes.

Specifically, the present inventors found that 2-deoxyglucose uptake in adipocytes in culture is increased if the cells are treated with either dark roast coffee (naturally high in N-MP), coffee spiked with N-MP, or N-MP as purified compound compared to control cells. Accordingly, in one embodiment, the present invention provides a method for preventing or treating diabetes, such as improving glucose uptake in adipocytes or muscle cells of a subject in need thereof, comprising administering to the subject a pharmaceutical composition comprising an effective amount of isolated N-methylpyridinium, or a pharmaceutically acceptable derivative thereof, and a pharmaceutically acceptable excipient.

The term “derivative” as used herein, includes all compounds based on N-MP suitable for human or animal consumption for food, drink, or health or wellness purposes that have the same physiological/pharmaceutical effect. As such, all active pharmaceutical ingredients (API's) having N-MP as core structure and having the same physiological/pharmaceutical effect are covered by this invention.

Thus, the term “derivative”, as mentioned herein, in particular is directed to compounds defined by the following

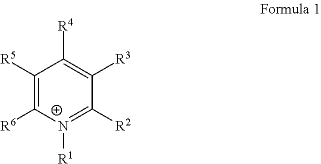

The substituents R¹, R², R³, R⁴, R⁵ and R⁶ may be defined in the broadest possible way, under the proviso that R¹ is at least methyl (or a substituent having a longer chain).

For example, they may be selected from hydrogen, substituted or unsubstituted aliphatic or aromatic hydrocarbons, such as alkyl, alkenyl, alkinyl, cycloalkyl, hydroxyalkyl, alkoxy, phenyl, benzyl groups and derivatives thereof; from halogen (Cl, Br, F, I), NO, CN, NO₂, OH, SH, NH₂, carboxyl, or aldehyde, just to name a few. Based on the information that the derivative must contain a N-MP core structure and general chemical considerations such as steric hindrance etc., the person having average skill in the field of designing API's will be capable, based on in vitro experiments such as those disclosed herein, to determine whether the one or the other derivative will fall within the scope of the present invention or not. That is to say, whether it possesses an N-MP core structure and whether it has a capacity to prevent or treat type II or I diabetes, for example based on 2-deoxyglucose uptake in adipocytes. According to the present invention, such a capacity is defined as any enhancement of the 2-deoxyglucose uptake in adipocytes.

In a preferred embodiment, the substituents of formula 1 are defined as follows:

R¹ is selected from a branched or linear alkyl or hydroxyalkyl chain of C₁-C₂₂. Further, R², R³, R⁴, R⁵ and R⁶ are independently selected from hydrogen, a branched or linear alkyl or hydroxyalkyl chain of C₁-C₂₂ or a carboxyl (—COOH) group. Encompassed are also pharmaceutically acceptable salts thereof.

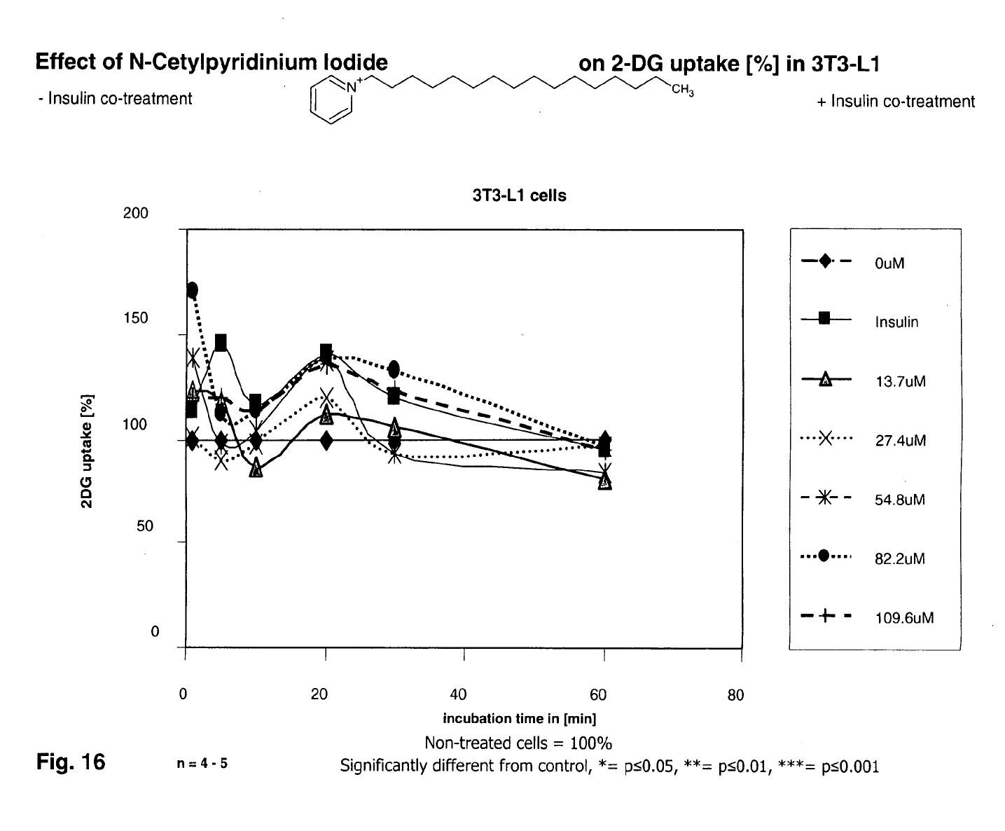

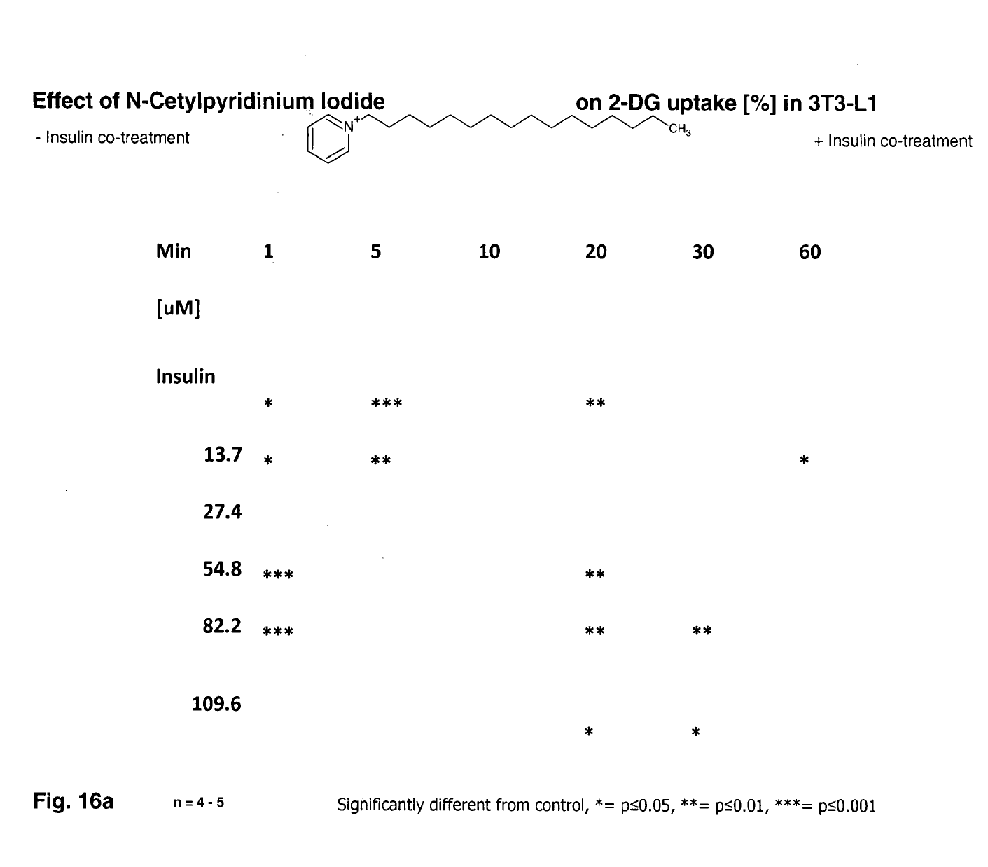

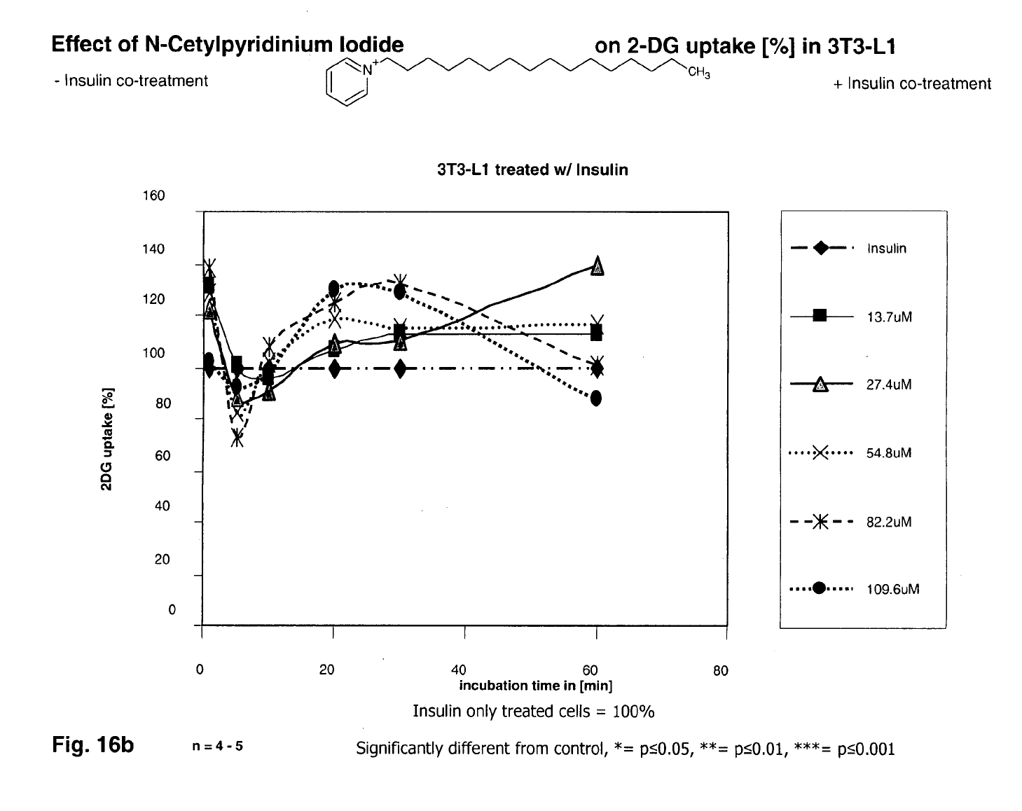

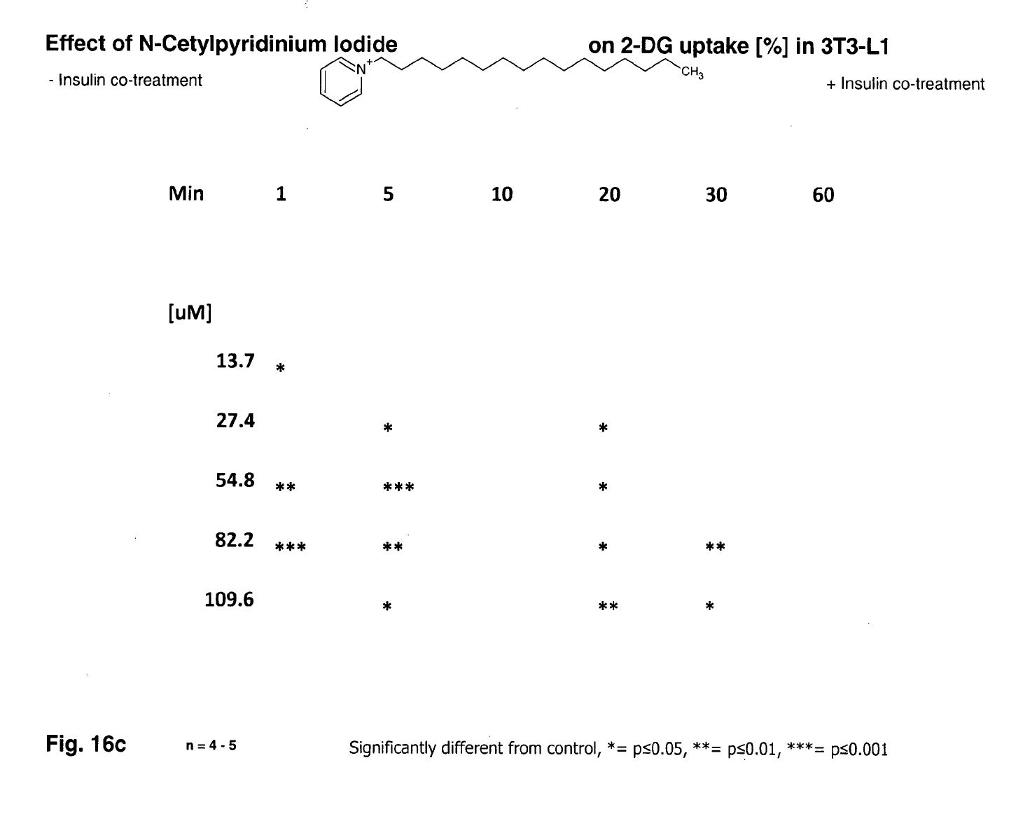

It is noted that the best results and effects are achieved if R¹ is C₁ (=methyl) and each of R², R³, R⁴, R⁵ and R⁶ are H (see formula 8=N-MP). However, also longer alkyl substituents at R¹ showed enhanced 2-deoxyglucose uptake in adipocytes in culture and thus an improved effect in the prevention/treatment of type II or I diabetes. Preferred examples of longer alkyl substituents for R¹ are methyl, ethyl and cetyl. For example, N-cetylpyridinium iodide showed excellent effects on the uptake of 2-deoxyglucose, whether used alone or in combination with insulin (see FIG. 16).

Furthermore, good results could be achieved for N-ethylpyridinium iodide and it is further envisioned, that also other alkyl substituents within the frame of C₁-C₂₂ are suitable derivatives of N-methylpyridinium for the medical use envisioned in the present invention.

As mentioned above, substituents R², R³, R⁴, R⁵ and R⁶ can be selected from hydrogen, a branched or unbranched alkyl or hydroxylalkyl chain of C₁-C₂₂ or as an alternative, from a carboxyl group. Also here, it is preferred if R², R³, R⁴, R⁵ and R⁶ is C₁ (=methyl). However, bearing in mind the above comments, also longer alkyl chains are included in the scope of the present invention, for example C₂, C₃ or C₄.

Preferably, all of the substituents of R², R³, R⁴, R⁵ and R⁶ are hydrogen, or also preferred, four of them are hydrogen and the remaining one is selected from a branched or unbranched alkyl or hydroxyl alkyl chain of C₁-C₂₂. In more preferred embodiments, this remaining substituent is C₁ (=methyl).

Among the most preferred compounds of the present invention, there are two different groups of derivatives. The first group is based on derivatives of N-MP having substituents in the 2, 3 or 4 position of the aromatic ring. The second group is based on substituents in the R¹ position being longer than C₁.

Examples of the first group:
The compound according to Formula 1, wherein R¹ and R⁴ are methyl and R², R³, R⁵ and R⁶ are hydrogen (see Formula 2; N-4 methylpicolinium).

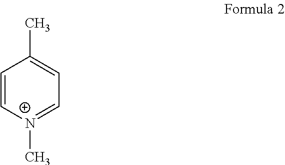

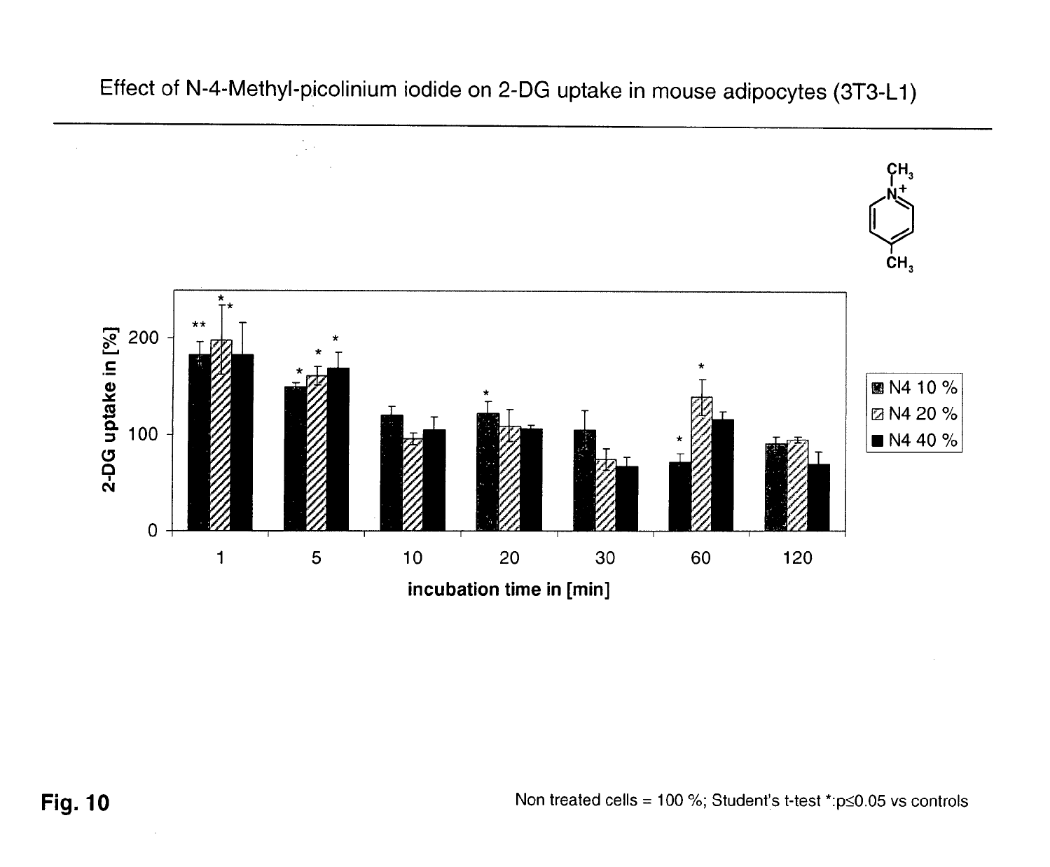

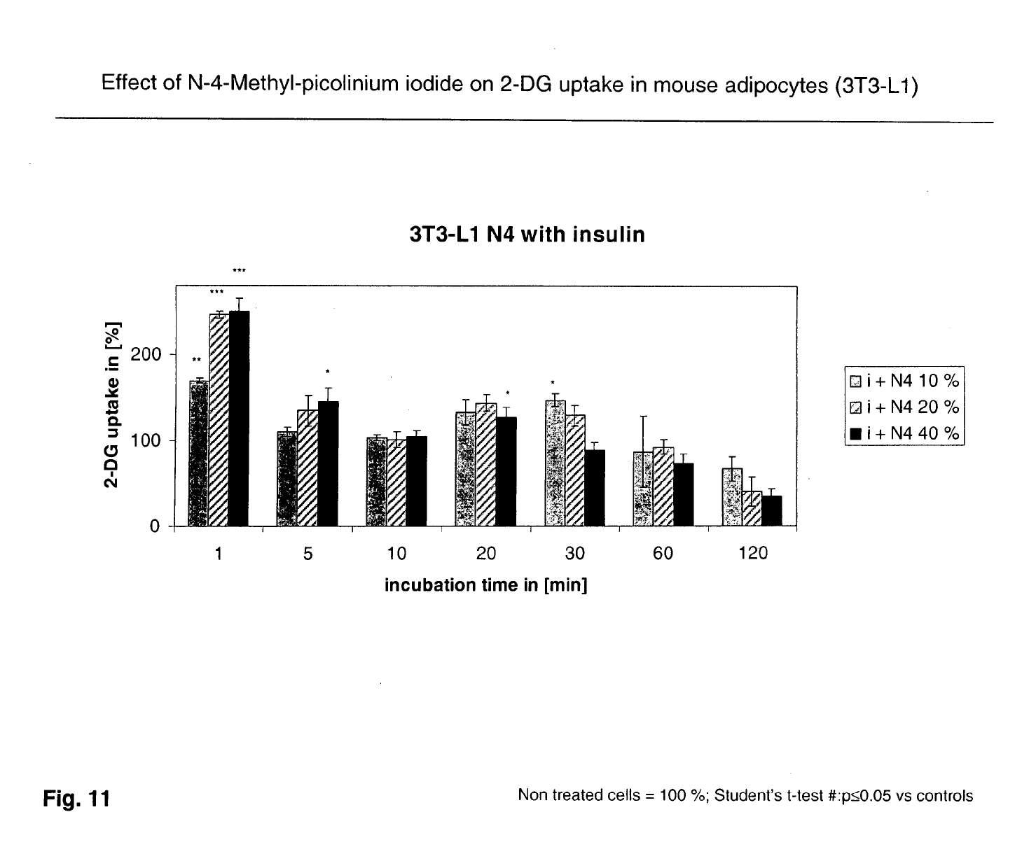

As it can be seen from FIGS. 10-14, N-4-Methylpicolinium in its iodide salt form shows a substantial increase in 2-deoxyglucose (2-DG) uptake whether used alone or in combination with insulin (see FIGS. 10 and 11). Independently from the concentration used, the 2-DG uptake is much higher than 100% and, in some cases, exceeds the value of 200% (see FIG. 11). It is noted that the highest results for the 2-DG uptake can be achieved in the first ten minutes of the incubation time which reflects the most important parameter also for the in vivo uptake of 2-DG by, for example, human body cells.
Further, a very active derivative is N-3-Methylpicolinium iodide (see Formula 3), wherein the results achieved are not as superior than those for N-4-Methylpicolinium iodide, however, still show a substantial improvement over the usual uptake of 2-DG.

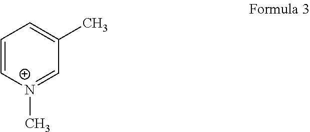

Also preferred is N-2-Methylpicolinium according to Formula 4:

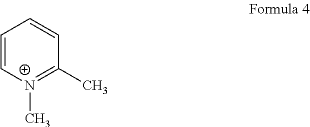

A still further, but less preferred embodiment of Formula 1 is trigonellin according to Formula 5, wherein R¹ is CH₃ is H, R³ is carboxyl and R², R⁴, R⁵ and R⁶ are H.

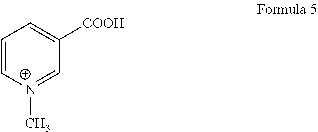

Generally, it is assumed that a substituent in the 4-position of Formula 1 provides better in vitro and in vivo activity, than substituents in the 3- or 2-position.

Examples of the second group:

As mentioned above R¹ is selected from C₁-C₂₂ alkyl or hydroxyalkyl.
A preferred embodiment is the compound according to Formula 6, i.e. Formula 1 wherein R¹=C₁₆, and R², R³, R⁴, R⁵ and R⁶ each are hydrogen.

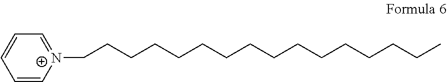

FIG. 16 shows the results of using N-cetylpyridinium iodide alone (left panel) and of co-treatment with insulin (right panel). In both applications, excellent results could be achieved in the decisive time frame of the first ten minutes after incubation. It is noted that this superior effect of N-cetylpyridinium iodide can be achieved nearly independently from the concentration used.
A further embodiment is the compound according to Formula 7, i.e. Formula 1 wherein R¹=C₂, and R², R³, R⁴, R⁵ and R⁶ each are hydrogen.

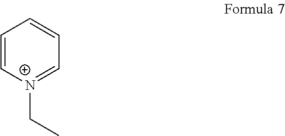

In one embodiment, the pharmaceutical composition is administered to the subject orally. In a preferred embodiment, the subject is a human.

In another embodiment, the present invention provides a method for treating or preventing type II or I diabetes in a subject in need thereof, comprising administering to the subject a pharmaceutical composition comprising an effective amount of isolated N-methylpyridinium, or a pharmaceutically acceptable derivative thereof, and a pharmaceutically acceptable excipient. Insulin is also administered to the subject, depending on the need of the subject. Preferably, the pharmaceutical composition of the present invention is administered to the subject orally.

The present invention provides in yet another embodiment a pharmaceutical composition comprising isolated N-methylpyridinium, or a pharmaceutically acceptable derivative thereof, and a pharmaceutically acceptable excipient.

The present invention further provides a food item, including a beverage, comprising isolated N-methylpyridinium or derivatives thereof. Preferably, the food item comprises an amount of N-methylpyridinium or of derivatives thereof effective for preventing type II or I diabetes. The beverage of the present invention may be, for example, coffee, tea, or beverages prepared therefrom, a soft drink, whether carbonated or not, drinking water, whether still or sparkling, a sport drink or energy drink, or even alcohol-containing beverage, such as cocktails, beer, or wine or hard liquor.

The most preferred embodiment of the present invention, i.e. N-methylpyridinium (N-MP), or N-methylpyridinium, or 1-methylpyridinium itself, has the following structure:

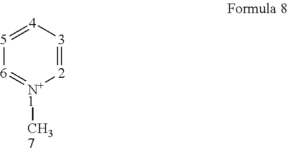

N-MP is known to naturally occur, or to exist in nature, e.g. in various amounts in roasted coffee. N-MP can be prepared by thermal treatment of trigonellin or trigonellin-rich sources or can be synthesized by methods well-known to those skilled in the art, see “Alkylpyridiniums. 1. Formation in Model Systems via Thermal Degradation of Trigonelline”, Richard H. Stadler, Natalia Varga, Jörg Hau, Francia Arce Vera, and Dieter H. Welti, J. Agric. Food Chem., 2002, 50 (5), pp. 1192-1199, which is incorporated herein by reference it its entirety.

The present invention is to, inter alia, compositions comprising isolated N-MP, or a pharmaceutically acceptable derivative thereof. As used herein, the term “isolated” refers to N-MP or a derivative thereof that is substantially free of other material with which it is normally found in nature, especially when it is substantially free of other naturally occurring cellular material. For example, “isolated N-MP” is free of caffeine and/or other ingredients found in roasted coffee.

Such isolated N-MP or derivatives thereof may be chemically synthesized, or enriched or otherwise isolated from a natural source. For example, prior art coffee beans, coffee beverages or other coffee products may comprise various concentrations of N-MP or derivatives thereof, depending, in part, on how or to what extent the coffee bean has been roasted. Such coffee beans, coffee beverages or coffee products are specifically excluded from the scope of the presently claimed invention. On the other hand, if N-MP or derivatives thereof, either chemically synthesized or otherwise obtained (e.g. by purification or enrichment methods from a natural product, such as roasted coffee), are added to the coffee bean, coffee beverages, or other coffee products, these coffee beans, coffee beverages, or coffee products with the aim of the product being used in the prevention or treatment of type I or type II diabetes mellitus would be considered to comprise “isolated N-MP or derivatives thereof” and would be within the scope of the presently claimed invention. Other food items, including other types of beverages and snacks are also within the scope of the present claimed invention.

As used in the context of the present invention, the term N-MP includes a pharmaceutically acceptable derivative of N-MP, including derivatives thereof suitable for human or animal consumption for food, drink, or health or wellness purposes that have the same physiological/pharmaceutical effect. Pharmaceutically acceptable N-MP derivatives include salts of N-MP, such as hydroxide, chloride, iodide, bromide, format, acetate salts, as well as the derivatives as outlined above and salts thereof. Furthermore, it should be noted that the pharmaceutical composition according to the present invention, in addition to N-methylpyridinium or the derivatives thereof as mentioned above may contain one or more pharmaceutically acceptable excipients.

The pharmaceutical composition of the invention may, in addition to NMP or derivatives thereof, contain one or more further active ingredients, which can enhance the overall activity of the composition or lower side effects thereof, for use in the treatment or prevention of diabetes type II or I.

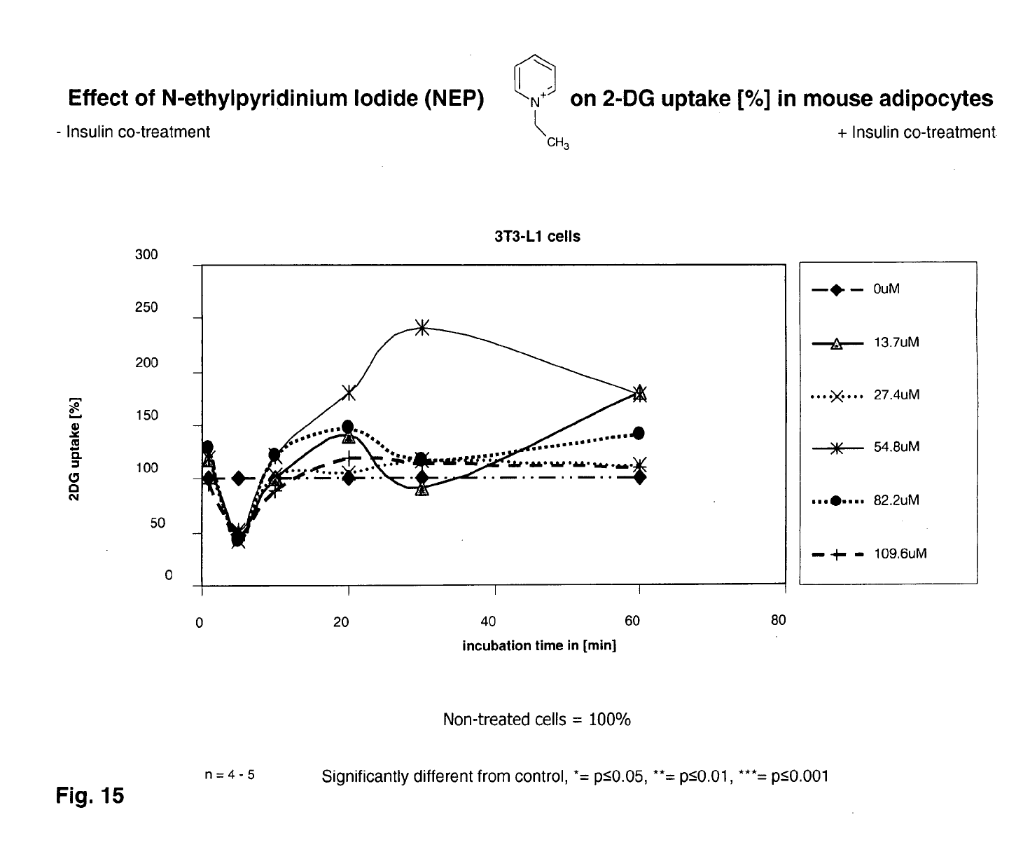

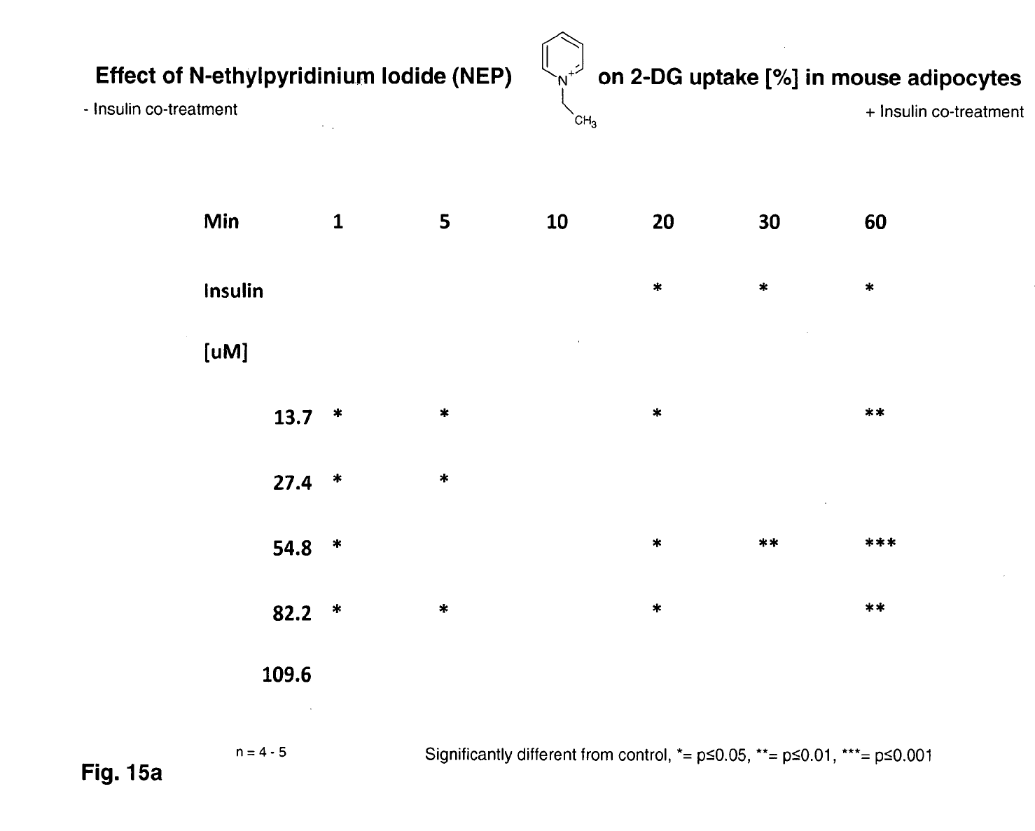

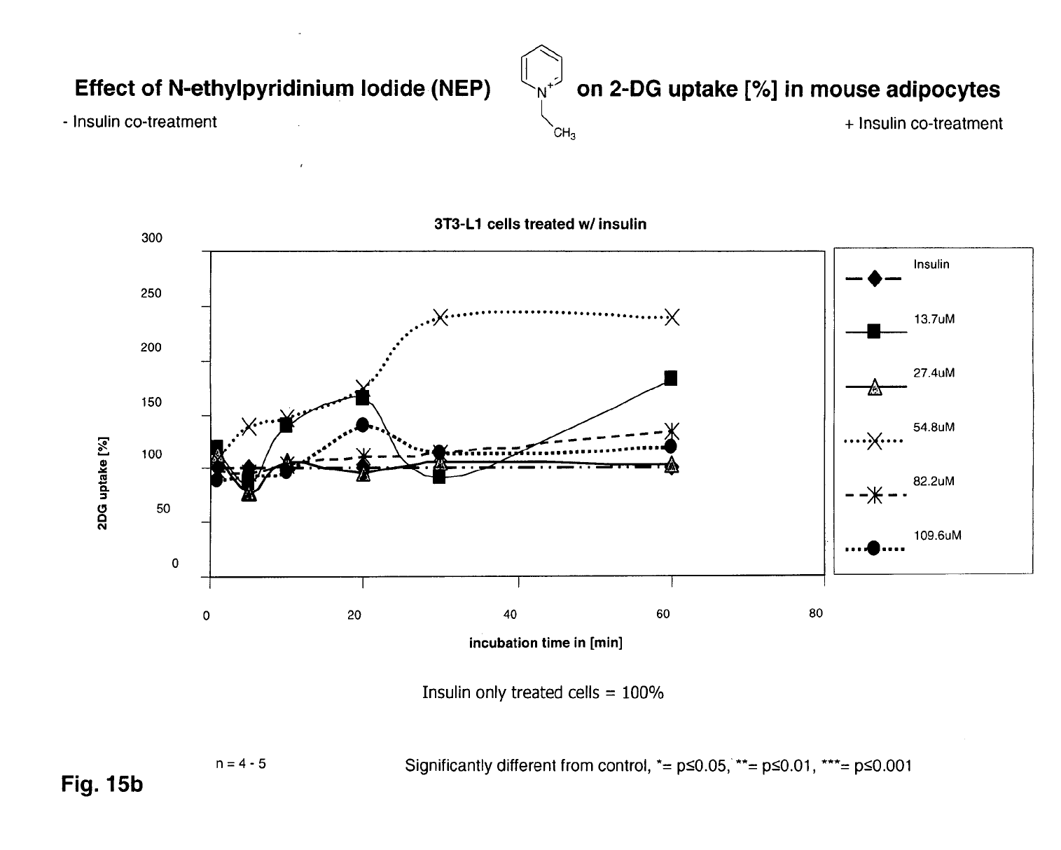

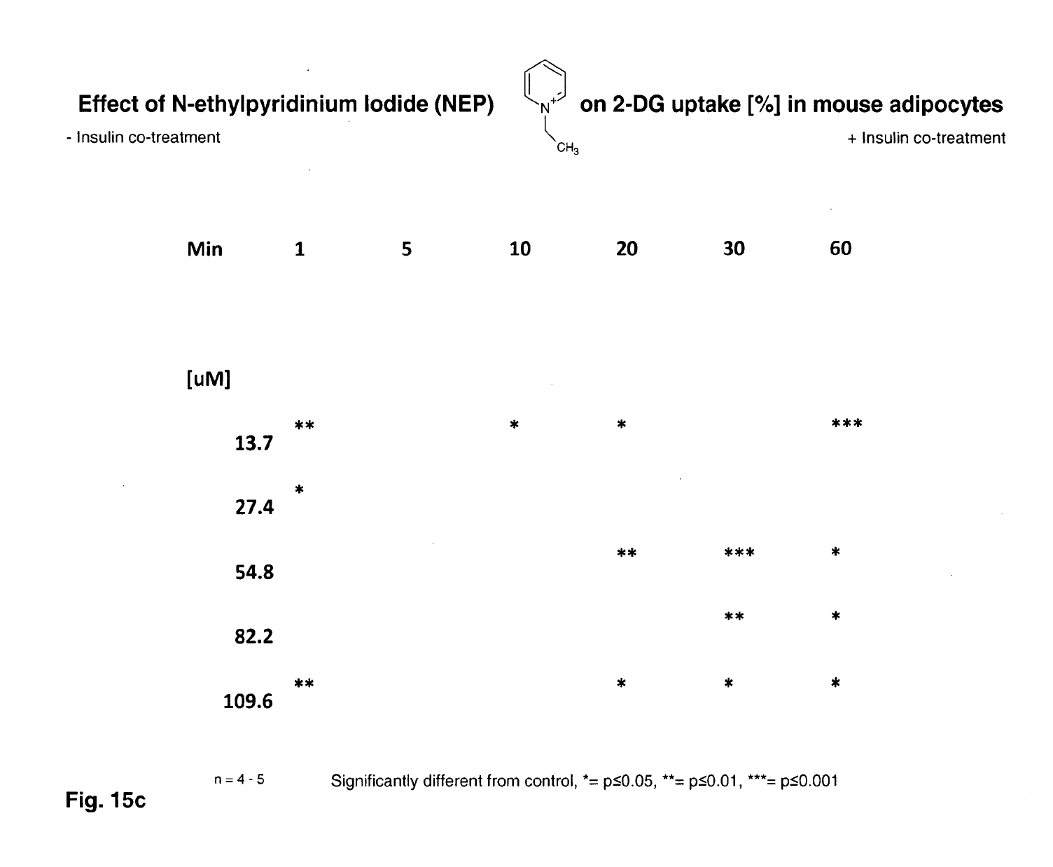

A preferred additional ingredient is insulin. As it can be seen from the enclosed examples and figures, the ingredients of the present invention can be readily combined with insulin thereby achieving further improved and/or synergistic results. See, for example FIG. 15 comparing the effect of the use of N-methylpyridinium iodide alone or in combination with insulin. According to the right panel of FIG. 15, it can be seen that the overall activity of the composition may be improved by this way.

Furthermore, there is the option to include other active ingredients which are usually contained in coffee or coffee extracts and which are not based on Formula 1, see above. Among others, substances from the group of catechols, chlorogenic acid and behenoyl-5 hydroxytryptamide can be named as ingredients, which are used along with the above-mentioned components together in one composition.

The pharmaceutical preparations of the present invention are manufactured in a manner which is itself known, for example, by means of conventional dissolving or suspending the compounds, which are all either water soluble or suspendable. The pharmaceutical preparations which can be used orally include push-fit capsules made of gelatin, as well as soft, sealed capsules make of gelatin and a plasticizer such as glycerol or sorbitol. The push-fit capsules can contain the active compounds in liquid form that may be mixed with fillers such as lactose, binders such as starches, and/or lubricants such as talc or magnesium stearate and, optionally, stabilizers. In soft capsules, the active compounds are preferably dissolved or suspended in suitable liquids, such as in buffered salt solution. In addition, stabilizers may be added.

In addition to being provided in a liquid form, for example in gelatin capsule or other suitable vehicle, the pharmaceutical preparations may contain suitable excipients to facilitate the processing of the active compounds into preparations that can be used pharmaceutically. Thus, pharmaceutical preparations for oral use can be obtained by adhering the solution of the active compounds to a solid support, optionally grinding the resulting mixture and processing the mixture of granules, after adding suitable auxiliaries, if desired or necessary, to obtain tablets or dragee cores

Suitable excipients are, in particular, fillers such as sugars, for example lactose or sucrose, mannitol or sorbitol, cellulose preparations and/or calcium phosphates, for example tricalcium phosphate or calcium hydrogen phosphate, as well as binders such as starch, paste using for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, crosslinked polyvinyl pyrrolidone, agar, or algenic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which if desired, are resistant to gastric juices. For this purpose, concentrated sugar solutions may be used, which may optionally containing gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye stuffs or pigments may be added to the tables or dragee coatings, for example, for identification or in order to characterize combinations of active compound doses.

Suitable formulations for parenteral administration include aqueous solutions of the active compounds. In addition, suspensions of the active compounds as oily injection suspensions may be administered. Aqueous injection suspensions may contain substances which increase the viscosity of the suspension and may include, for example, sodium carboxymethyl cellulose, sorbitol, and/or dextran. Optionally, the suspension may also contain stabilizers. Parenteral administration usually may be done by subcutaneous (s.c.), intravenous (i.v.), intramuscular (i.m.), or intraperitoneal (i.p.) administration.

If one or more of the active ingredients of the present invention are used in combination with insulin, it is conceivable that they are not administered as one single entity, but used as a combined medicament. For example, the active ingredients of the present invention are provided by oral administration, such as by a tablet or capsule, and insulin is provided in another way, i.e. in a parenteral way or by inhalation. Therefore, the invention also encompasses the combined application of two or more ingredients in different ways to a patient suffering from diabetes type II or I.

The active ingredients of the present invention should be administered in a suitable pharmaceutical composition such that they are applied in dosages ranging from 0.003 to 30.0 mg per kg of the patient's body weight per day, preferably from 0.05 to 5.0 mg per kg body weight per day. A most preferred dosage would be about 0.5 to 3 mg per kg body weight per day. For example, the daily dosage for an average human patient would amount to approximately 35 to 350 mg per day. Insulin or the other active ingredients which are optionally administered in combination with the ingredients of the present invention, will be applied according to the usual therapy plan established by the physician in charge.

### Examples

### Manufacturing Procedure

N-cetylpyridinium bromide and trigonelline hydrochloride, and all chemicals for synthesis were obtained from Sigma-Aldrich, Steinheim, Germany. All chemicals were of the highest purity available.

The iodide salts of N-methylpyridinium, N-methyl-2-picolinium and N-methyl-4-picolinium were synthesized using the protocol described by Stadler et al. (J. Agric. Food Chem., 2002, 50 (5), pp 1192-1199) with some modifications. Briefly, an excess of methyliodide (1.2 mmol) was added dropwise to a solution of pyridine (1 mmol), 2-picoline (1 mmol), or 4-picoline (mmol), respectively, in dry acetonitrile (5 mL) with stirring. The resulting solution was heated (reflux, 30 min), then left standing at room temperature for cooling and finally placed on ice for crystallization of the salt. The product was recrystallized from acetonitrile and kept under vacuum for storage.

N-methyl-3-picolinium iodide was prepared by heating methyliodide (1.2 mmol) with 3-picoline in dry acetonitrile (5 mL) as mentioned above, and subsequent treatment of the still warm solution with tert.butylmethylether (40 mL), yielding the target compound as an orange solid. The solid was filtered of, washed with portions of tert.butylmethylether and crystallized from dry acetonitrile and kept under vacuum for storage.

Trigonelline hydroiodide was prepared by refluxing nicotinic acid (1 mmol) with methyliodide (1.2 mmol) in ethanol (20 mL) as reported in the literature (Ciusa and Nebbia, Ciusa, W.; Nebbia, G. The preparation of salts of N-methylnicotinic acid. Gaz. Chim. Ital. 1950, 80, 98-99). After evaporation of the solution, the residue was crystallized twice from ethanol/water (95/5, v/v).

N-ethylpyridinium iodide was prepared by addition of methyliodide (2 mmol) to a solution of pyridine (1 mmol) in tert.butylmethylether (1 mL). The solution was vortexed and incubated at room temperature for 2 days. Finally, the resulting suspension was kept at −20° C. (5 h) prior to centrifugation and removal of the supernatant. The residue was washed with tert.butylmethylether, dried by lyophilisation (48 h, 0.77 mbar, 25° C.) and kept under vacuum for storage.

### Analytical Procedure

Mouse adipocytes (cell line 3T3-L1) as well as mouse myotubes (cell line C2C12) were cultivated under standard conditions and treated either with regular cell culture medium or insulin for 4 hours. Afterwards, cells were exposed to a combination of 2-deoxyglucose (2-DG) and the respective sample, either N-MP, coffee beverage (content of NMP in the coffee beverage: 26.7 mg/L) or a combination of both, for 2 hours. Then, cells were harvested and the 2-DG uptake was analyzed photometrically using the resorufin assay (Yamamoto, N.; Sato, T.; Kawasaki, K.; Murosaki, S.; Yamamoto, Y. A nonradioisotope, enzymatic assay for 2-deoxyglucose uptake in L6 skeletal muscle cells cultured in a 96-well microplate. Analytical Biochemistry 2006, 351, 139-145).

The experimental protocol is outlined as follows:

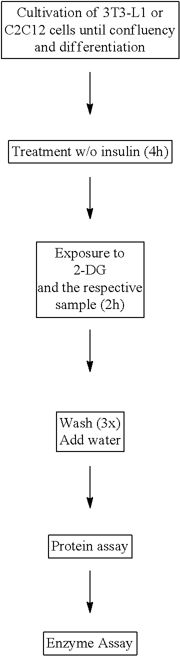

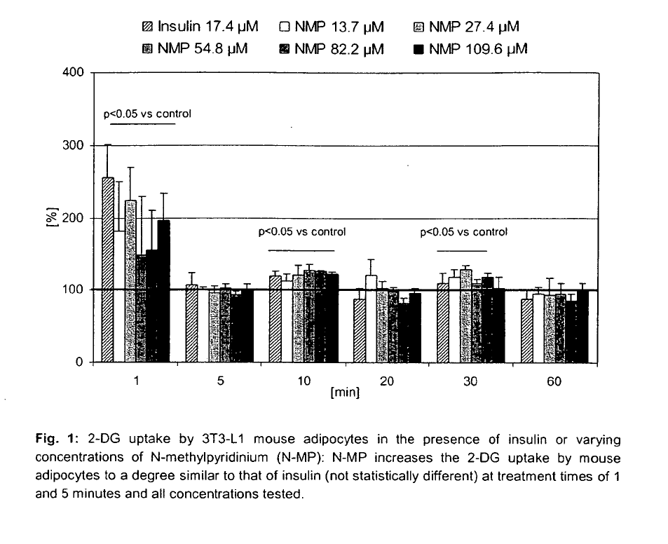

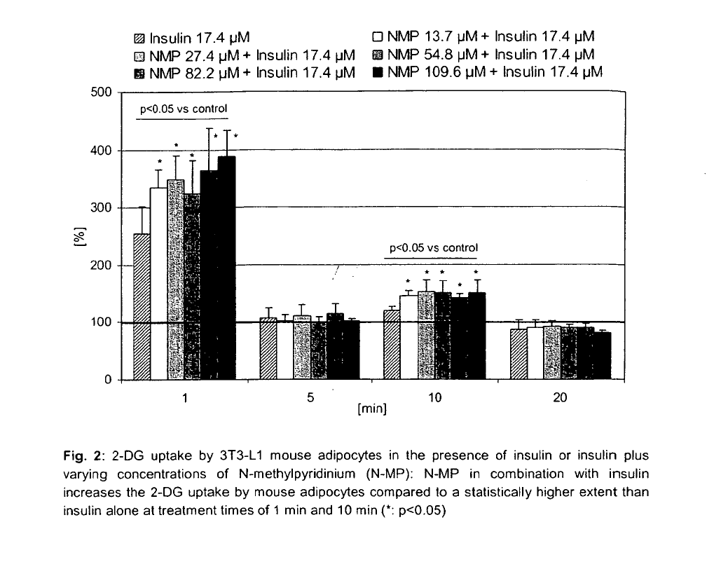

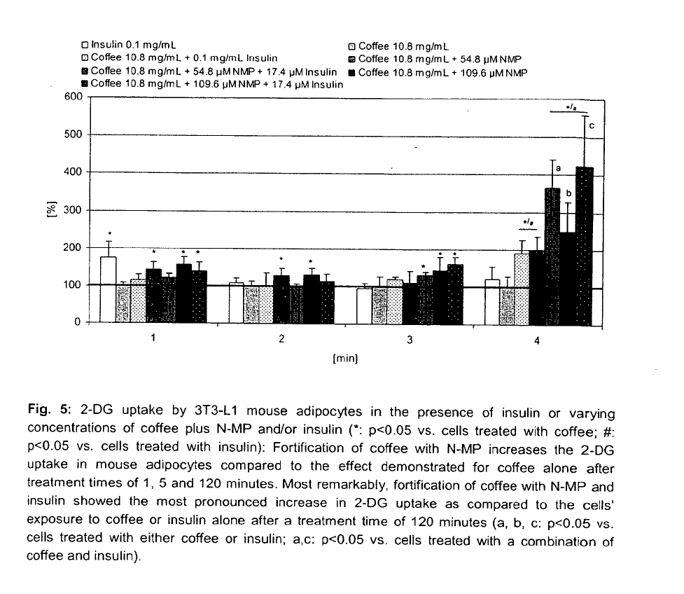

The results show that N-Methylpyridinium and derivatives thereof enhance the uptake of 2-deoxyglucose in mouse adipocytes similarly insulin (FIG. 1), significantly enhance the effect of insulin (FIG. 2) and, in combination with coffee beverage, greatly enhance the effectiveness of insulin (FIG. 5).

More specifically, FIG. 1 shows that N-MP increases the 2-DG uptake by mouse adipocytes to a degree similar to that of insulin (not statistically different) at treatment times of 1 and 5 minutes and at varying concentrations tested.

FIG. 2 shows that N-MP in combination with insulin increases the 2-DG uptake by mouse adipocytes to a statistically higher extent than insulin alone at treatment times of 1 min and 10 min.

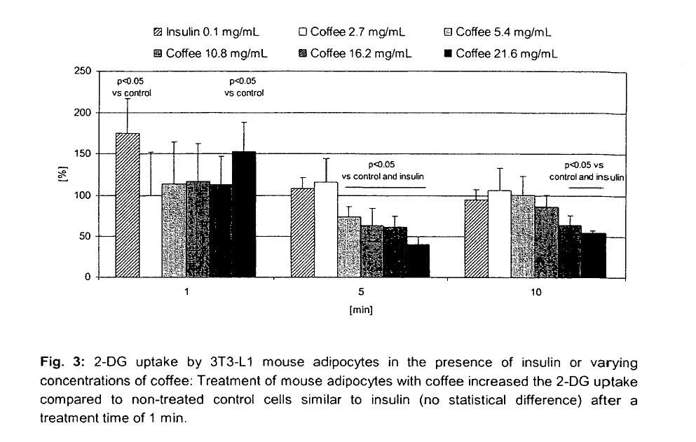

FIG. 3 shows that treatment of mouse adipocytes with coffee increased the 2-DG uptake compared to non-treated control cells similar to insulin (no statistical difference) after a treatment time of 1 min.

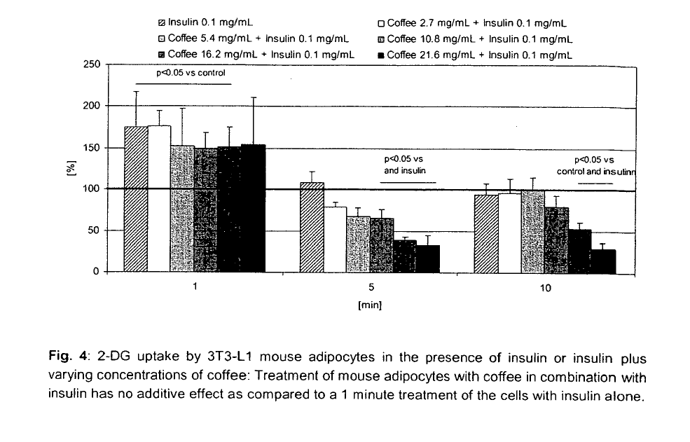

FIG. 4 shows that treatment of mouse adipocytes with coffee in combination with insulin has no additive effect as compared to a 1 minute treatment of the cells with insulin alone.

FIG. 5 shows that fortification of coffee with N-MP increases the 2-DG uptake in mouse adipocytes compared to the effect demonstrated for coffee alone after treatment times of 1, 5 and 120 minutes. Most remarkably, fortification of coffee with N-MP and insulin showed the most pronounced increase in 2-DG uptake as compared to the cells' exposure to coffee or insulin alone after a treatment time of 120 minutes.

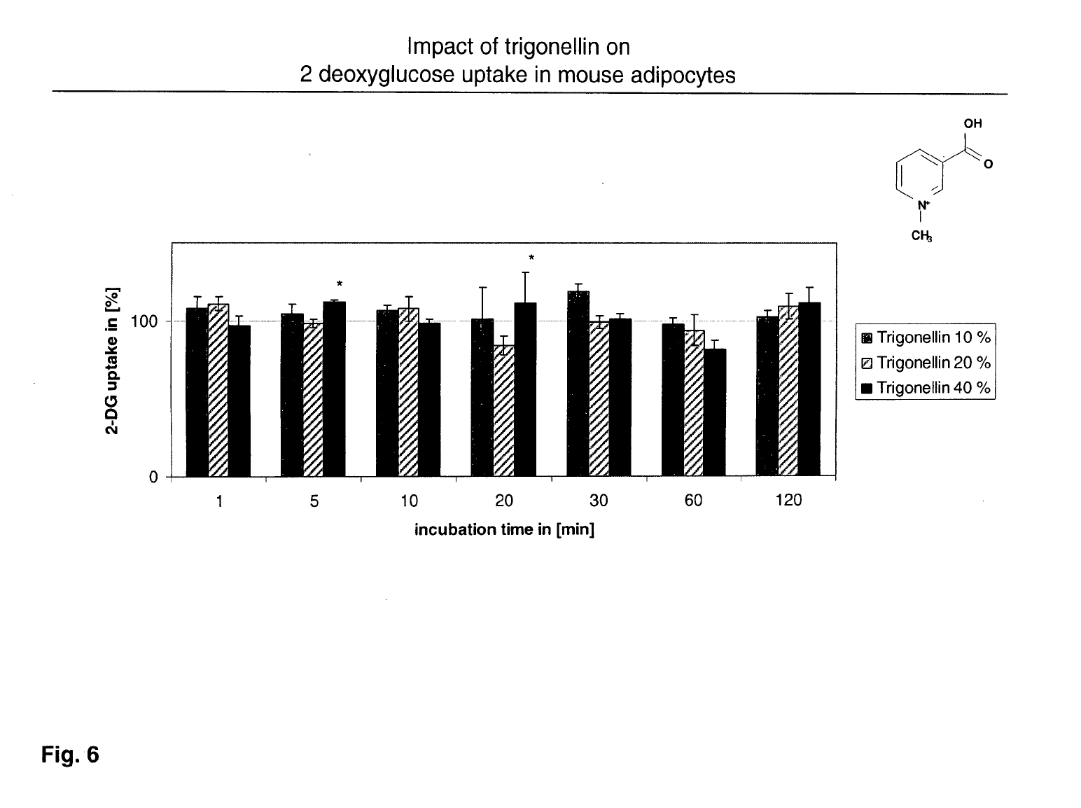

FIG. 6 shows the impact of trigonellin on 2 deoxyglucose uptake in mouse adipocytes.

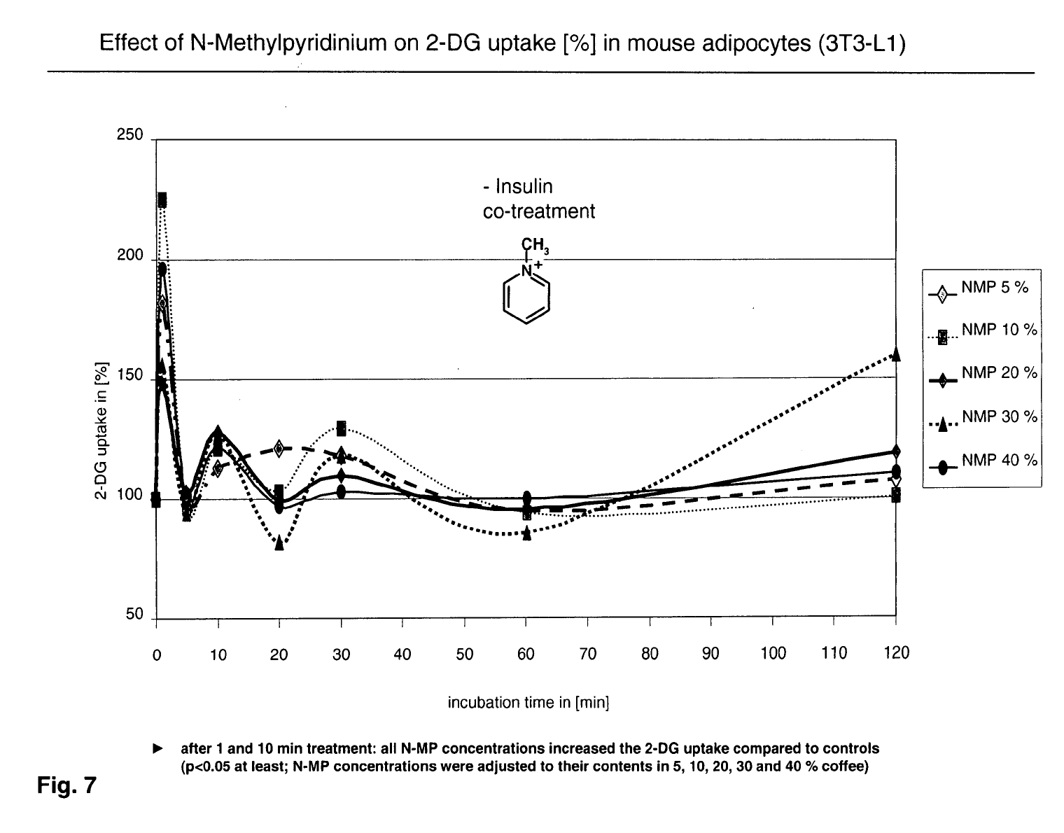

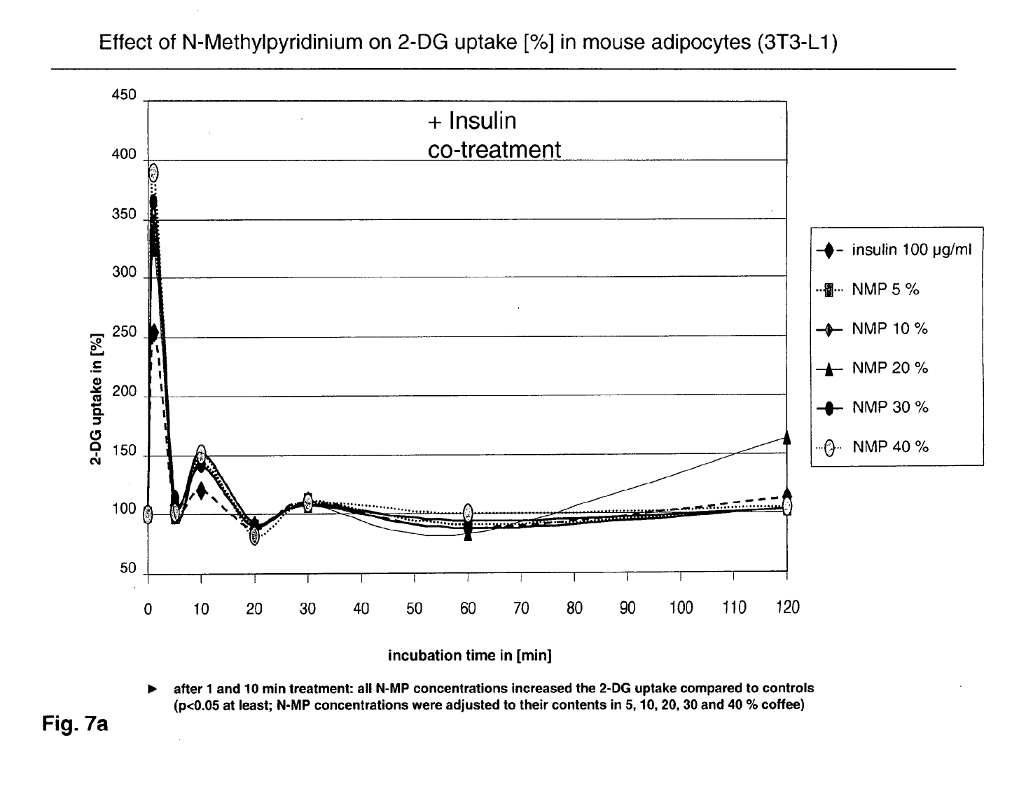

FIG. 7 shows the effect of N-Methylpyridinium on 2-DG uptake [%] in mouse adipocytes (3T3-L1). The upper panel depicts the results for N-Methylpyridinium alone, the lower panel shows the results for N-Methylpyridinium in combination with insulin.

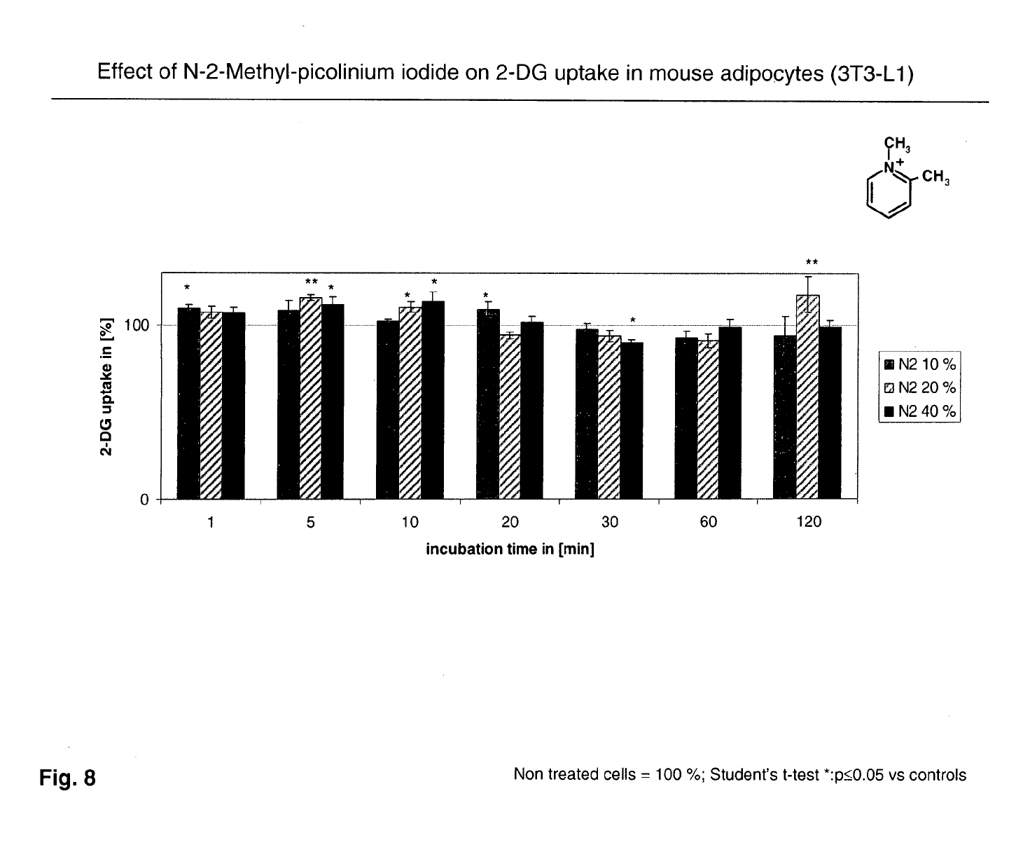

FIG. 8 shows the effect of N-2-Methyl-picolinium iodide on 2-DG uptake in mouse adipocytes (3T3-L1).

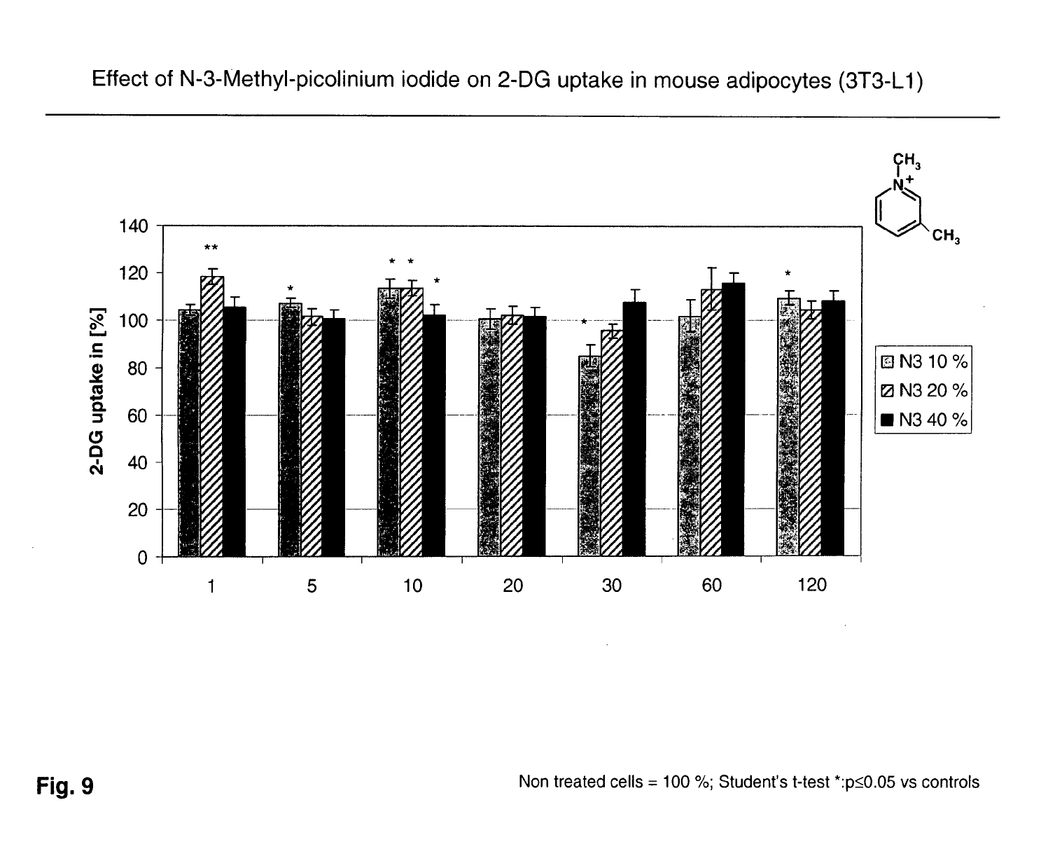

FIG. 9 is a graph showing the effect of N-3-Methyl-picolinium iodide on 2-DG uptake in mouse adipocytes (3T3-L1).

FIG. 10 describes the effect of N-4-Methyl-picolinium iodide on 2-DG uptake in mouse adipocytes (3T3-L1) without the concurrent use of insulin.

FIG. 11 describes the effect of N-4-Methyl-picolinium iodide on 2-DG uptake in mouse adipocytes (3T3-L1) with the concurrent use of insulin.

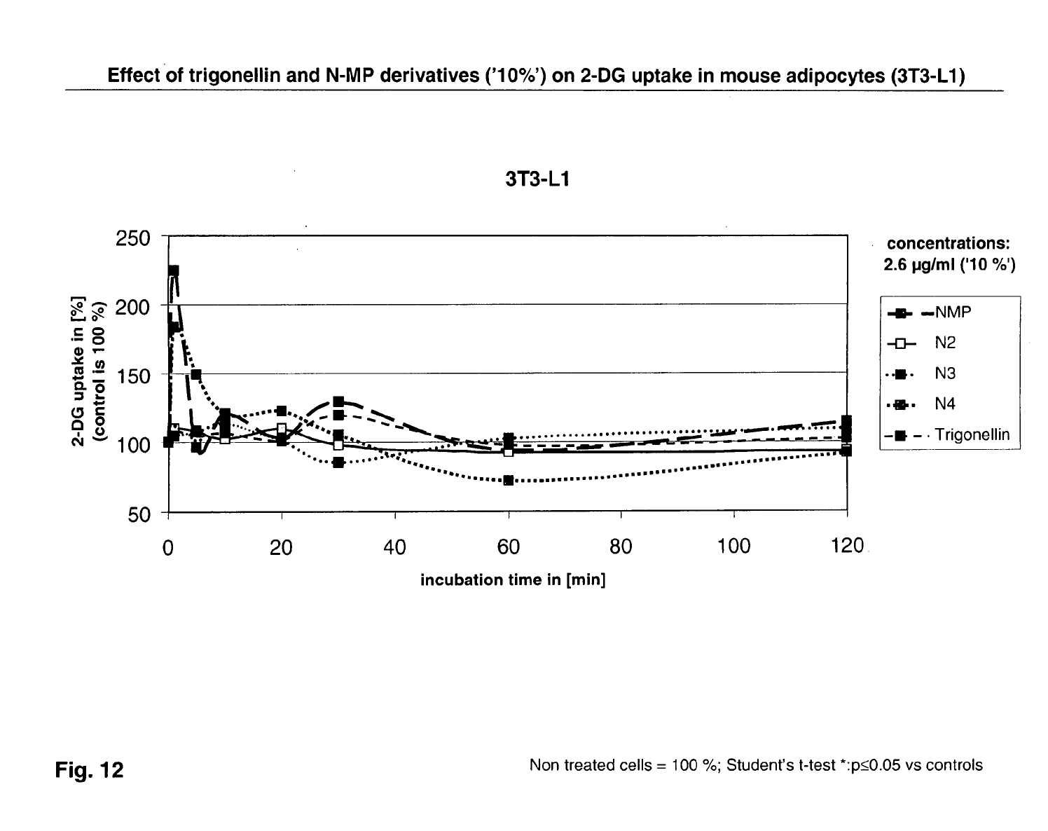

FIG. 12 shows the effect of trigonellin and N-MP derivatives (‘10%’) on 2-DG uptake in mouse adipocytes (3T3-L1).

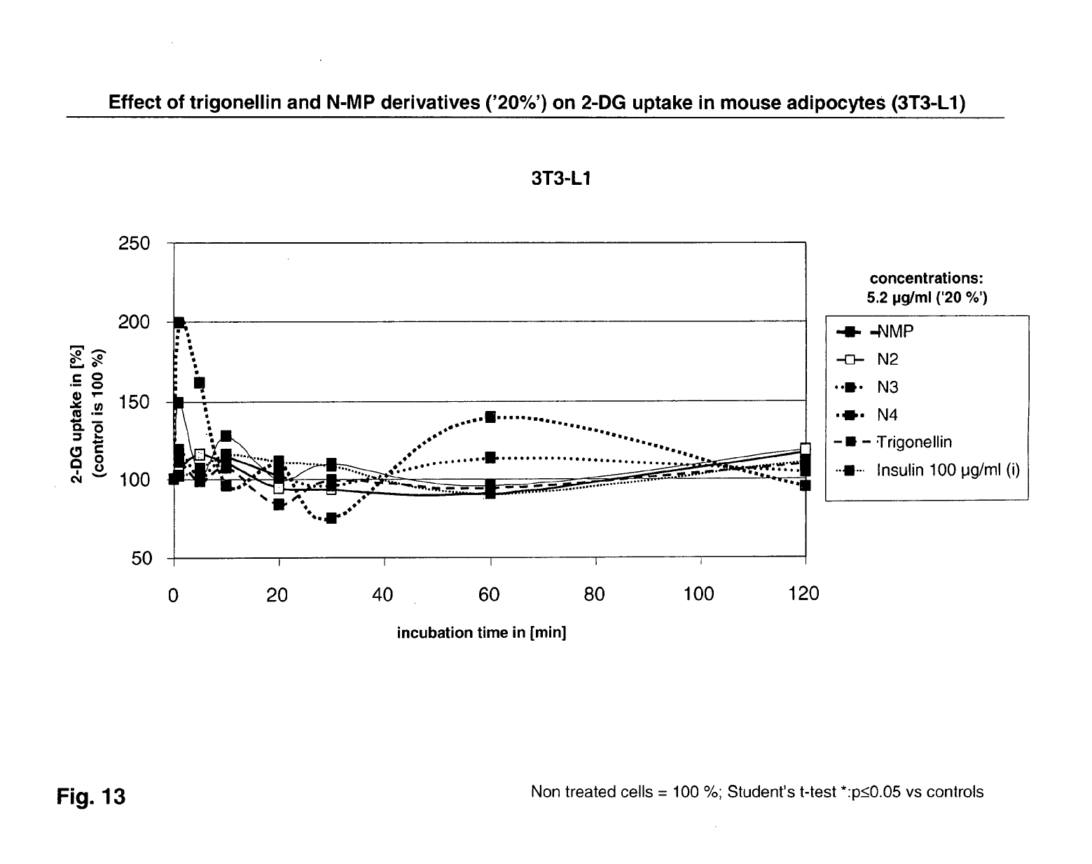

FIG. 13 shows the effect of trigonellin and N-MP derivatives (‘20%’) on 2-DG uptake in mouse adipocytes (3T3-L1).

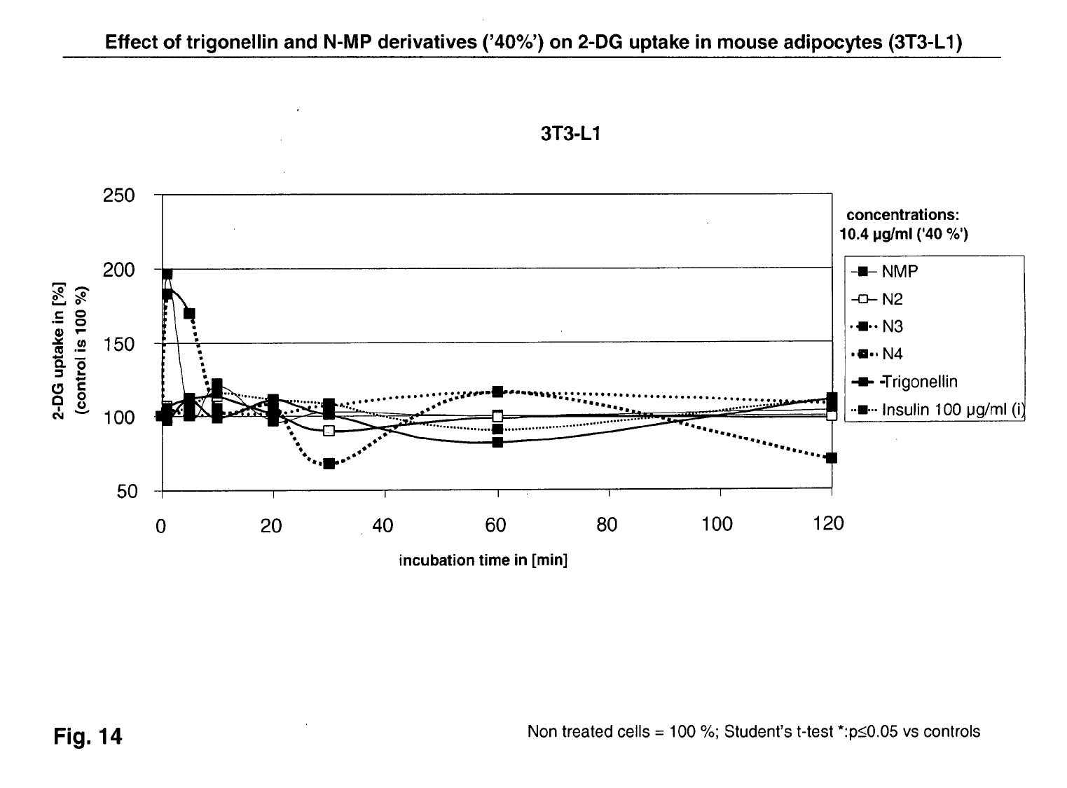

FIG. 14 shows the effect of trigonellin and N-MP derivatives (‘40%’) on 2-DG uptake in mouse adipocytes (3T3-L1).

FIG. 15 describes the effect of N-ethylpyridinium iodide (NEP) on 2-DG uptake [%] in mouse adipocytes. Left panel: no insulin cotreatment. Right panel: insulin cotreatment.

FIG. 16 shows the effect of N-Cetylpyridinium Iodide on 2-DG uptake [%] in 3T3-L1. Left panel: no insulin cotreatment. Right panel: insulin cotreatment.

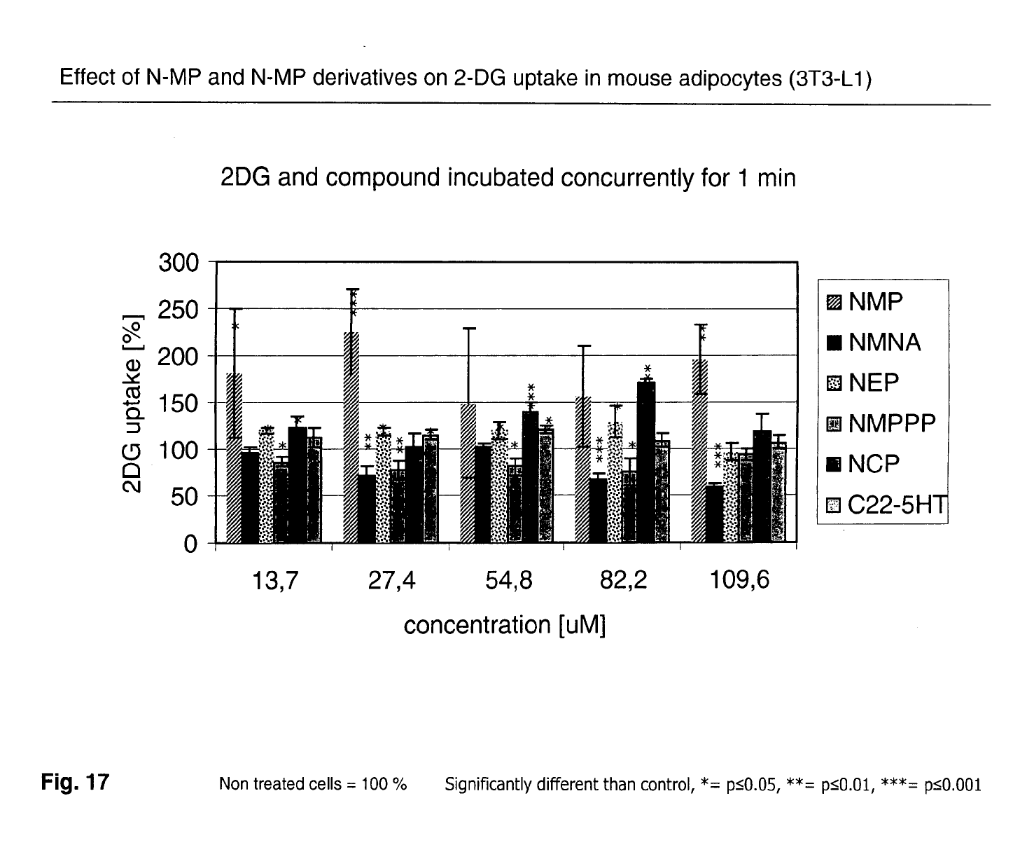

FIG. 17 shows the effect of N-MP and N-MP derivatives on 2-DG uptake in mouse adipocytes (3T3-L1).

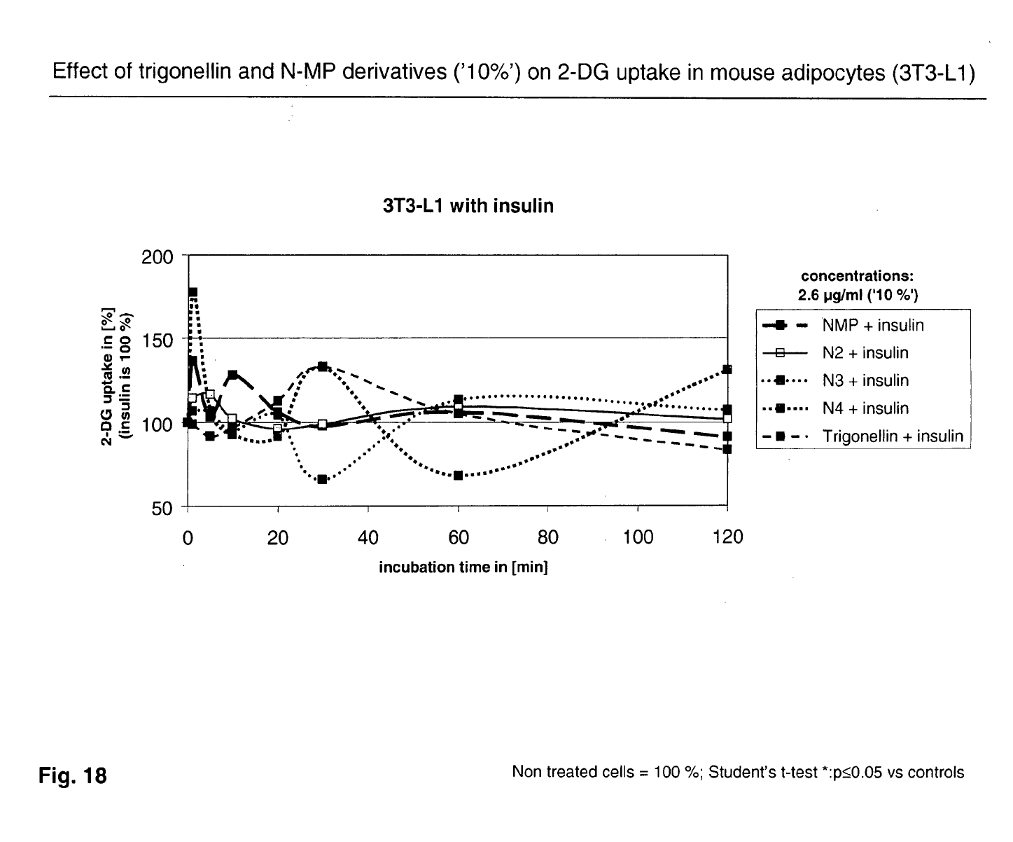

FIG. 18 shows the effect of trigonellin and N-MP derivatives (‘10%’) on 2-DG uptake in mouse adipocytes (3T3-L1).

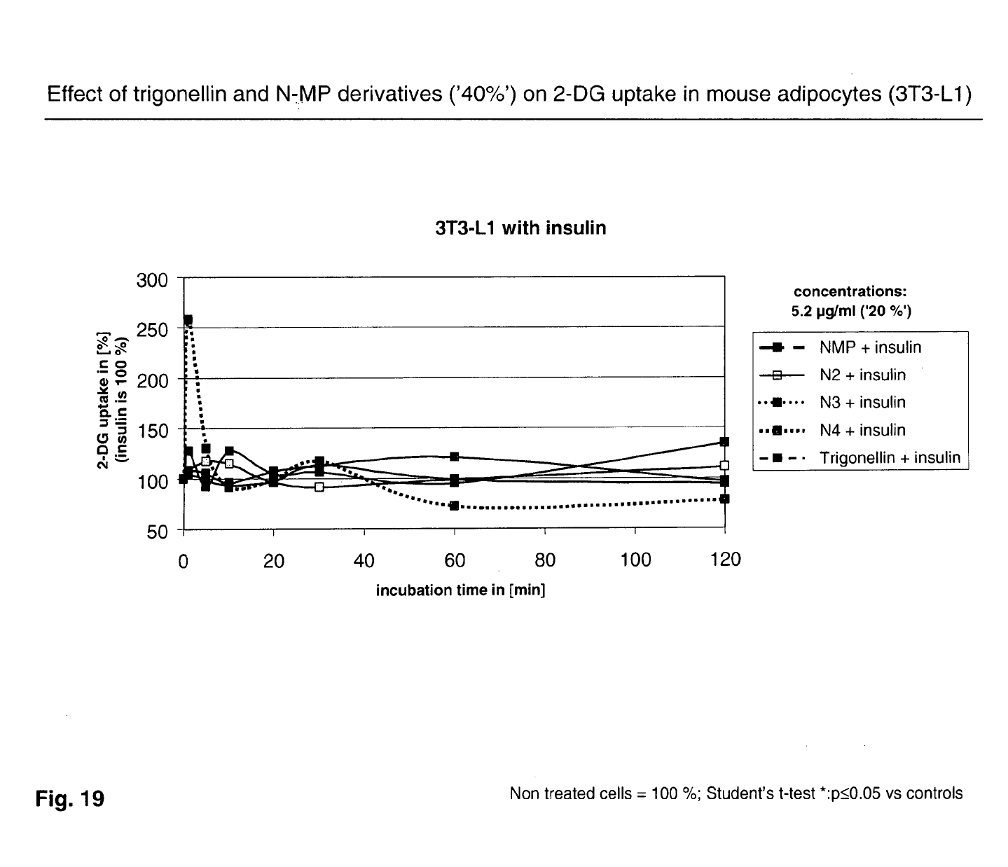

FIG. 19 shows the effect of trigonellin and N-MP derivatives (‘20%’) on 2-DG uptake in mouse adipocytes (3T3-L1).

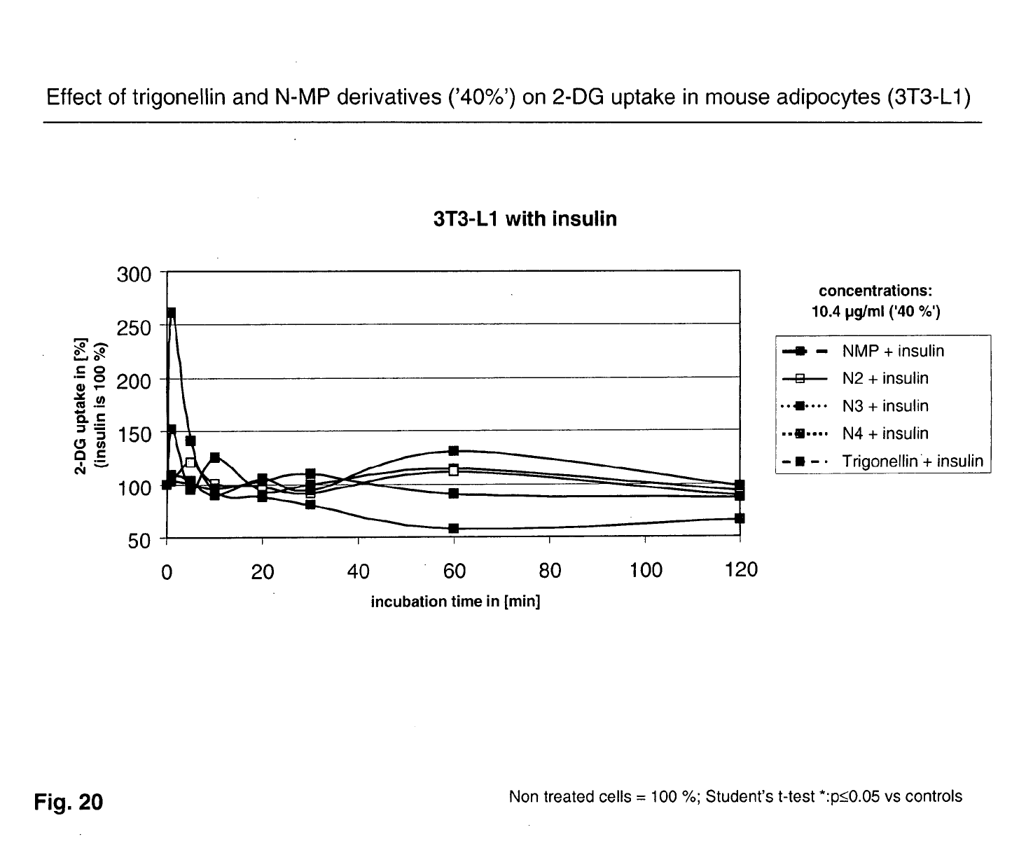

FIG. 20 shows the effect of trigonellin and N-MP derivatives (‘40%’) on 2-DG uptake in mouse adipocytes (3T3-L1).

1.-18. (canceled)

19. A pharmaceutical composition comprising isolated N-methylpyridinium, or a pharmaceutically acceptable derivative thereof, and a pharmaceutically acceptable excipient.

20. The pharmaceutical composition of claim 19, wherein the derivative is defined by 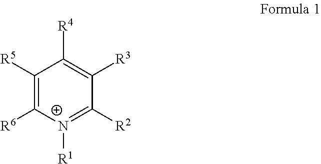
wherein R¹ is selected from a branched or linear alkyl or hydroxyalkyl chain of C₁-C₂₂; and
R², R³, R⁴, R⁵ and R⁶ are independently selected from hydrogen, a branched or linear alkyl or hydroxyalkyl chain of C₁-C₂₂ or a carboxyl (—COOH) group, or a pharmaceutically acceptable salt thereof.

21. The pharmaceutical composition of claim 19 or 20, which contains further active ingredients, in particular insulin.

22. The pharmaceutical composition of claim 19 for use in the prevention or treatment of type II or I diabetes.

23. A food item comprising isolated N-methylpyridinium or a pharmaceutically acceptable derivative or salt thereof, as defined in claim 19.

24. The food item of claim 23, which comprises an amount of N-methylpyridinium or a pharmaceutically acceptable derivative or salt thereof effective for preventing type II or I diabetes.

25. A beverage suitable for human consumption comprising isolated N-methylpyridinium or a pharmaceutically acceptable derivative or salt thereof.

26. The beverage of claim 25, which comprises an amount of N-methylpyridinium or a pharmaceutically acceptable derivative or salt thereof effective for preventing type II or I diabetes.

27. The beverage of claim 26, which is selected from the group consisting of coffee, tea, carbonated soft drink, still water, sparkling water, sport drink, and alcohol-containing beverage.

28. A method for improving glucose uptake in adipocytes or muscle cells of a subject in need thereof, comprising administering to the subject a pharmaceutical composition comprising an effective amount of isolated N-methylpyridinium, or a pharmaceutically acceptable derivative thereof as defined in claim 19, and a pharmaceutically acceptable excipient.

29. The method according to claim 28, wherein the pharmaceutical composition is administered to the subject orally.

30. The method according to claim 28, wherein the subject is a human.

31. A method for treating or preventing type II or I diabetes in a subject in need thereof, comprising administering to the subject a pharmaceutical composition comprising an effective amount of isolated N-methylpyridinium, or a pharmaceutically acceptable derivative thereof as defined in of claim 19, and a pharmaceutically acceptable excipient.

32. The method according to claim 31, wherein type II diabetes is prevented.

33. The method according to claim 31, wherein type II diabetes is treated.

34. The method according to claim 31, wherein insulin is also administered to the subject.

35. The method according to claim 31, wherein the pharmaceutical composition is administered to the subject orally.

36. The method according to claim 31, wherein the subject is a human.
